(19) United States
(12) Patent Application Publication
(10) Pub. No.: US 2019/0175843 A1
(43) Pub. Date: Jun. 13, 2019

(54) PORT NEEDLE

(71) Applicant: TRINOGY UG (HAFTUNGSBESCHRÄNKT), Unterföhring (DE)

(72) Inventor: Markus MASIN, Riga (LV)

(73) Assignee: TRINOGY UG (HAFTUNGSBESCHRÄNKT), Unterföhring (DE)

(21) Appl. No.: 16/300,486

(22) PCT Filed: May 25, 2016
(86) PCT No.: PCT/EP2016/025046

§ 371 Date: Nov. 9, 2018

(30) Foreign Application Priority Data
May 12, 2016 (EP) 16020177.8

(51) Int. Cl. A61M 5/32; A61L 31/02

(52) U.S. Cl. CPC A61M 5/329; A61M 5/3286; A61L 31/022

(57) ABSTRACT

The present invention is directed to a port needle that is adapted to penetrate skin, tissue and/or a septum of a port to transfer fluid into a port chamber of the port, the port needle comprising at least one lumen defining at least one inner surface, the lumen being adapted to transfer the fluid from the distal section to the proximal section and at least one membrane defining at least one outer surface of the proximal section, wherein the outer surface of the proximal section comprises nickel (Ni) and/or any Ni compound with a content of at most 0.05% and/or being adapted to release at most 0.2 μg/cm²/week of Ni and/or of any Ni compound.

### FIELD

The present invention is directed to a port needle that is particularly adapted to penetrate skin, tissue and/or a port membrane of an implanted port to transfer fluid into a port chamber of the port.

### BACKGROUND

Port needles or punctuation needles into port catheters often need to remain in a body of a patient for an extended period of time. Up to seven days the needles remain in the body.

Port needles are hollow needles designed to apply liquids into a prior implanted port or port catheter are commonly used and known. Various constructions can be easily or safely used by medical personal.

While often syringe-type devices are utilized for vascular access ports in acute or short-term situations, a special type of device is utilized for longer term infusion therapy. Such an infusion assembly device generally consists of a needle and wing assembly that lies flat against the skin in an insertion position, the needle having a proximal end attached to a wing assembly that is in angular relation to the needle shank, the angle being approximately 90°. As mentioned, when this longer term needle device is inserted into the vascular access port, it lies flat against the patient's skin and is adhered thereto as described, for example, in U.S. Pat. No. 4,710,176, which is herewith incorporated by reference.

With respect to the needle tip of the needle used in infusion assemblies for vascular access ports, a non-coring configuration is generally utilized due to repeated entry of the needle into the septum or membrane of the vascular access port. When the tip of a hypodermic or other traditional needle advances through the membrane or septum, coring occurs if any portion of the septum material is forced inside the shaft of the needle through the opening in the tip thereof. That portion of the septum material forced inside a needle in this process is in effect severed from the rest of the body of the septum material. Such septum coring produces small, detached particles of the septum that are likely to enter the fluid that is infused by the implanted vascular access system into the vascular system of the patient. These particles can obstruct fluid flow through the outlet stem of the vascular access port, or if escaping through the outlet stem of the vascular access port, can become trapped in the cardiovascular system of the patient. In addition, septum coring produces small passageways through the body of a septum. On occasion these passageways extend entirely through the septum, from the exterior thereof to the fluid reservoir inside the vascular access port.

The inwardly directed forces imposed on the installed septum by the housing of a vascular access port should initially urge the material of the body of the septum inwardly upon itself to close such passageways after the shaft of the needle is withdrawn therefrom. Nonetheless, continued coring eventually leads to various forms of septum failure that cannot be overcome by such inwardly directed forces. The material continuity of the septum is increasingly compromised, resulting in crumbled areas of the septum matrix.

Eventually, leakage of fluid can be expected through the septum from the fluid reservoir in the vascular access port. Once such fluid escapes to the exterior of the vascular access port, necrosis will occur of the tissue surrounding the subcutaneous packet in which the vascular access port is implanted, causing many undesirable consequences. Therefore, non-coring or Huber needles are preferably used in conjunction with infusion assemblies for vascular access ports. These needles, in contradistinction to the standard or traditional hypodermic needles pierce the septum like a knife, facilitating the resealing thereof so that the aforementioned problems are largely averted.

As with any needle-type device, there exists the problem of inadvertent needle sticks, which generally occurs when the needle-type device is withdrawn from the patient prior to appropriate disposal thereof. Of course, inadvertent needle sticks introduce a variety of concerns due to unwanted transmission of blood from the patient to the medical practitioner. Inadvertent needle sticks can occur because of carelessness on the part of the medical practitioner or due to accidents in the handling of devices with exposed needle tips. With respect to Huber needles specifically, needle stick accidents can occur due to difficulty in removal as well. This difficulty results from the configuration of the Huber needle, which gets hooked into the port making it difficult to remove. Increased pulling force on behalf of the medical practitioner to dislodge the needle from the port results in less control over the tip of the needle when freed from the port, causing the inadvertent needle stick.

To address the inadvertent needle stick problem in winged infusion assemblies, such as those described herein, various safety devices have been designed to encase the needle after it is withdrawn from the port. One such safety device is described in U.S. Pat. No. 5,755,694 that discloses a needle base disposed over a segment of the needle at its proximal end, comprising two generally flat wings made of flexible material with one hinge connecting each of the wings to the needle base. Upon removal of the needle from the patient the wings flex against a moveable member that keeps them adjacent the needle until the needle is completely removed from the skin of a patient after which the wings surround the needle to prevent the tip from making contact outside of the needle base. This patent is herewith incorporated by reference.

Another type of safety device is described in U.S. Pat. No. 5,951,522 in which the configuration of the Huber needle is described. Patent '522 discloses a safety enclosure comprising a wing assembly mounted to the aft end of an angled Huber needle, the wing assembly having a configuration consisting of either a single integral member having a plurality of spaced apart fold lines which divide the member into a plurality of interconnected panels, or a pair of wing members mounted in a scissors-type arrangement. In each embodiment, when the medical practitioner removes the needle from the patient, the wing assembly closes around the needle and locks together, encasing it therein. This patent is herewith incorporated by reference.

A further invention has disclosed a safety needle in U.S. application 2013/0218085, which describes a safety device for protecting a port needle or Huber needle against shifting. It permits particularly good fixing of the position of the port needle or Huber needle since that part of the port needle or Huber needle that lies on the surface of the skin of the patient is received in the inner opening of the protective device. The frame-like spacer element surrounds this part of the port needle or Huber needle lying on the surface of the skin of the patient and extends over a large part of the height of the needle. In this way, a kind of embankment is created which provides protection all the way round the port needle or Huber needle. This application is herewith incorporated by reference.

However, infections of unknown origin have been detected by the prior art devices, particularly at the location, where the port needle permeates the skin.

### SUMMARY

Port catheters are by principle always intended to feed medical substances into and/or retrieve fluids from a human or animal body. This therapy is named “parenteral therapy”, i.e. the transfer of the fluid in either direction circumvents the digestive system. A plurality of applications is commonly in practical use comprising parenteral vascular and/or arterial medication with all appropriate and approved infusion therapies, parenteral intrathecal medication with all appropriate and approved infusion therapies, parenteral peritoneal medication with all appropriate and approved infusion therapies, parenteral intraspinal or epidural medication with all appropriate and approved infusion therapies, constant negative aspiration of ascites and/or any combinations thereof.

The present invention is specified in the claims as well as in the below description. Preferred embodiments are particularly specified in the dependent claims and the description of various embodiments.

The present invention is directed to a port needle that is particularly adapted to penetrate skin, tissue and/or a port septum of a port to transfer fluid into a port chamber of the port.

The port can optionally be provided to be operated outside a body of a human or an animal. The fluid is then transported just into the chamber, e.g. for simulation purposes and/or training purposes.

The port needle comprises a distal section adapted to receive the fluid and a proximal section adapted to deliver the fluid to the chamber of the port. The fluid can be received from any reservoir, container etc. containing any appropriate kind of fluid and/or material for a patient. The fluid will then be fed or conveyed to the proximal section. The proximal section can be the section directly communicating with a port or being adjacent the port. It also comprises the section penetrating the port's septum and/or the skin and/or tissue of a patient.

At least one lumen can define or form at least one inner surface, the lumen being adapted to transfer the fluid from the distal section to the proximal section. More than one lumen for different purposes can also be provided. The purposes can be cooling, heating, normalizing temperatures, feeding other fluids and/or retrieving fluids. The port needle can then be designed to also provide the respective structures for all these functions or just parts of them.

At least one membrane defines at least one outer surface of the proximal section. The membrane can be a solid and/or rigid wall and/or any layer or coating on a solid and/or rigid substrate. A substrate can be a wall as well on which then the membrane is arranged, preferably in a fixed manner. In case of one lumen the wall forming the membrane or the substrate can have round cross-section or any other form, such as square or polygonal or any combination thereof. In case of more than one lumen, the outer wall can also have such shapes in accordance with the arrangement of the lumens.

The outer surface of the proximal section comprises a metal, forming a compound or alloy containing nickel (Ni) with a maximum content of 0.05% and/or being adapted to release at most 0.2 μg/cm²/week of Ni and/or of any Ni compound. The content of Ni and/or any compound and/or the capability to release Ni and/or any compound can also be 0 (zero) or get practically close to 0 (zero).

The membrane can be impermeable to liquids and/or gases. This depends on the fluids used and also the constitution of the wall or substrate. In case the latter are impermeable the membrane may be permeable to fluids but for sterility reasons an impermeability is preferred so that no fluid and unwanted materials, bacteria, germs etc. can easily rest.

The membrane can be arranged as at least one outer layer on a substrate and/or is forming an outer wall at least of the proximal section. The membrane can be made of metal and/or metal alloy, such as steel, preferably stainless steel. Alternatively, at least the membrane of the proximal portion of the port needle or the entire port needle comprises and/or is made of a non-ferric metal or a non-metallic material and preferably comprises carbon and/or CFRP materials.

The section modulus of the port needle is at least 0.004 mm³, preferably 0.01 mm³, more preferably 0.1 mm³, even more preferably 0.2 mm³ and/or at most 0.3 mm³, preferably 0.2 mm³, more preferably 0.1 mm³, even more 0.01 mm³. This is meaningful, because a port needle device, even if it is attached to a patient, and the needle itself is placed in a port chamber, carries the risk of breakage. Fracturing of the needle, when in use, could lead to splinters that could potentially carry lethal risk for the patient.

The membrane can be defined by a coating and/or a layer provided over at least the proximal section of the port needle, preferably adhered by a shrink fit and/or an adhesive.

The inner surface of the port needle can comprise Ni or any Ni compound of at most 0.05% and/or being adapted to release at most 0.2 μg/cm²/week of Ni or of any Ni compound. The content of Ni and/or any compound and/or the capability to release Ni and/or any compound can also be 0 (zero) or get practically close to 0 (zero).

The proximal section can have a maximum length of 50 mm, preferably of 40 mm, more preferably of 30 mm, even more preferably of 20 mm, and even more preferably of 10 mm and/or a minimum length of 2 mm, preferably of 5 mm, more preferably of 10 mm, even more preferably of 20 mm, even more preferably of 30 mm.

The solid membrane and/or the membrane arranged as a layer together with the underlying substrate has/have a maximum outer diameter of 1.5 mm, preferably of 1.2 mm, more preferably of 1.0 mm, even more preferably of 0.9 mm, even more preferably of 0.8 mm, even more preferably of 0.7 mm and/or a minimum diameter of 0.3 mm, preferably of 0.4 mm, more preferably of 0.5 mm, even more preferably of 0.6 mm, even more preferably of 0.7 mm, even more preferably of 0.9 mm.

Theoretically port needles are available from 6 G till 30 G. Nowadays needles are generally used having the sizes 22 G, 21 G, 20 G and 19 G (G meaning the gauge identifying specific inner diameters). 19 G is commonly used for blood, albumin, thrombocyte concentrates etc. Sizes of 20 to 22 are commonly used for common or uncommon infusion fluids, such as chemotherapeutic compositions, total and/or partly parenteral feeding, also named parenteral nutrition, antibiosis, virustasis, antibody-therapies, etc.

The proximal section of the port needle can terminate in a blunt end, a bevelled end and/or an end with at least one lateral window.

The proximal section can comprise a dull end and the port needle can further comprise a piercing element with a sharp tip being invertible at least into the lumen so that the sharp tip extends out of the proximal section for penetrating the skin, tissue and/or port lumen and the piercing element being adapted to be taken out of the lumen. Such piercing element is also called a mandrain.

The invention is also directed to a needle for the medical use as a port needle as specified before and below.

The present invention also relates to a method of penetrating skin, tissue and/or a port septum of a port by a port needle to transfer fluid into a port chamber of the port. The method can also be practiced for the penetration of a port septum of a port outside the human or animal body.

The present invention can comprise the following steps: receiving the fluid by a distal section; delivering the fluid to the chamber of the port by a proximal section; transferring the fluid from the distal section to the proximal section by at least one lumen defining at least one inner surface; defining at least one outer surface of the proximal section by at least one membrane; and providing the outer surface of the proximal section with a Ni and/or any Ni compound with a content of at most 0.05% and/or the outer surface being adapted to release at most 0.2 μg/cm²/week of Ni and/or of any Ni compound.

The word “coated” is intended to comprise a process to be described as plated or metallized, electroplated, sputtered, shrinked, glued, adhered etc.

The word “body” and/or “organism” is intended to comprise the human and/or any warm-blooded animal body.

The word “fluid” is intended to also comprise liquids, gases, emulsions and/or any combination thereof.

The word “thorn” comprises a punctuation thorn, penetration tip, rod, mandrain, etc.

The word “membrane” comprises at least one sheath, film, lamina and/or covering layer and/or any combination thereof.

The word “compound” also comprises an alloy, mixture, combination, blend and/or any combination thereof.

The word “skin” also comprises cutaneous, subcutaneous tissue, subcutaneous fat, muscle.

It has been surprisingly found that allergic reactions can be avoided to a large extent by a modification of the port needle giving patients great relief.

### BRIEF DESCRIPTION OF THE DRAWINGS

The skilled person will understand that the drawings, described below, are for illustration purposes only. The drawings are not intended to limit the scope of the present teachings in any way.

FIG. 1 shows an example of a port needle arrangement 1 or port needle 1 from the bottom. At the side the distal section 17 can be seen receiving any fluids. A body 20 is holding all components or is arranged around at least part of them and may assist in holding them in place. At the body 20 an optional resting arrangement or resting layer 21 can be seen. A resting layer may be provided as an antibiotic means. Moreover, a proximal section 10 of the port needle 1 is shown that extends perpendicular to the paper plane. A grip 18 can be integrated for convenience purposes.

FIG. 2 exemplifies a side view of the port needle 1. The same or similar elements bear the same reference numerals. An optional resting portion 21 is arranged at the bottom of the port needle the resting portion 21 being provided to rest adjacent the port (not shown) or the skin and/or tissue in close proximity to the port. The proximal section 10 extends down, preferably in a perpendicular orientation or in a generally downward orientation.

FIG. 3 is further showing the port needle 1 with a septum 2. In addition to the figure before a punctuation thorn 25 is shown that can penetrate through the needle-septum 2, the thorn 25 having a sharp end 19. The sharp end 19 is brought into a position extending beyond the end of the proximal section 10 optionally having a blunt end 11. The thorn 25 assists in penetrating a port-septum 31 of a port 3, shown below, and can be retreated upon insertion of the port needle 1 in the port 3. The proximal end 10 can then rest in the port without a sharp end and may have a safe and more stable position therein. As the blunt end may bluntly rest against the bottom of a port chamber 30 a window 11′ can be provided ensuring flow of fluid out of the proximal section 10 through the window 11′.

FIG. 4 shows an optional end 11 of proximal section 10. The before disclosed membrane 13 can be solid and can define a lumen 12 with an interior surface 14 of the membrane 13 and an outer surface 15 of the membrane 13. In case the membrane is a layer or coating on a substrate, the membrane will define the outer surface 15 while the substrate will define the inner surface 14.

FIG. 5 shows two alternatives for designs of ends of the proximal sections 10 that also may be combined with each other. A window 11′, an inclined or truncated end or an inclined end with a window 11″ can be provided either alone or in combination. The embodiment of the proximal section 12′ can be formed as a “Huber needle” and/or any other non-coring needle-tip.

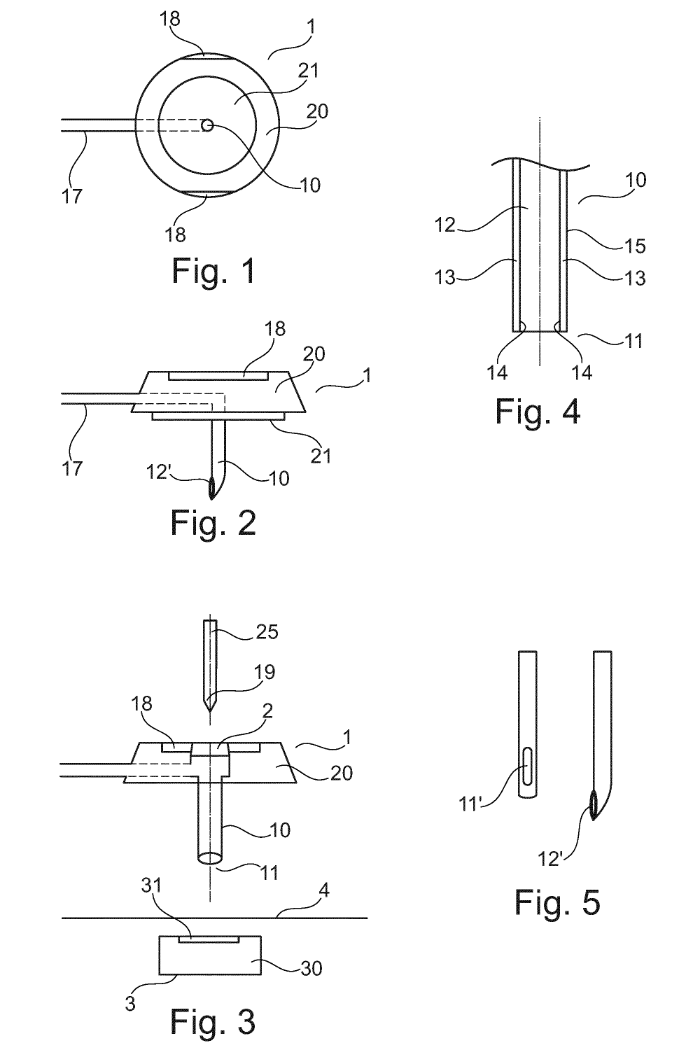

### DESCRIPTION OF VARIOUS EMBODIMENTS

In the following, exemplary embodiments of the invention will be described, referring to the figures. These examples are provided to provide further understanding of the invention, without limiting its scope.

In the following description, a series of features and/or steps are described. The skilled person will appreciate that unless required by the context, the order of features and steps is not critical for the resulting configuration and its effect. Further, it will be apparent to the skilled person that irrespective of the order of features and steps, the presence or absence of time delay between steps, can be present between some or all of the described steps.

As used herein, including in the claims, singular forms of terms are to be construed as also including the plural form and vice versa, unless the context indicates otherwise. Thus, it should be noted that as used herein, the singular forms “a”, “an” and “the” include plural references unless the context clearly dictates otherwise.

Throughout the description and claims, the terms “comprise”, “including”, “having”, and “contain” and their variations should be understood as meaning “including but not limited to”, and are not intended to exclude other components.

The present invention also covers the exact terms, features, values and ranges etc. in case these terms, features, values and ranges etc. are used in conjunction with terms such as about, around, generally, substantially, essentially, at least etc. (i.e., “about 3” shall also cover exactly 3 or “substantially constant” shall also cover exactly constant).

The term “at least one” should be understood as meaning “one or more”, and therefore includes both embodiments that include one or multiple components. Furthermore, dependent claims that refer to independent claims that describe features with “at least one” have the same meaning, both when the feature is referred to as “the” and “the at least one”.

It will be appreciated that variations to the foregoing embodiments of the invention can be made while still falling within the scope of the invention. Alternative features serving the same, equivalent or similar purpose can replace features disclosed in the specification, unless stated otherwise. Thus, unless stated otherwise, each feature disclosed represents one example of a generic series of equivalent or similar features.

The abbreviation “Ni” stands for the element nickel and/or comprising all isotope variations.

Use of exemplary language, such as “for instance”, “such as”, “for example” and the like, is merely intended to better illustrate the invention and does not indicate a limitation on the scope of the invention unless so claimed. Any steps described in the specification may be performed in any order or simultaneously, unless the context clearly indicates otherwise.

All of the features and/or steps disclosed in the specification can be combined in any combination, except for combinations where at least some of the features and/or steps are mutually exclusive. In particular, preferred features of the invention are applicable to all aspects of the invention and may be used in any combination.

The same reference numerals used for different embodiments are intended to identify parts or features of different embodiments with the same or similar function. In case the same reference numerals are not identified in other embodiments, this is by no means intended to mean that the corresponding features designated by these reference numerals are not present.

1. A port needle adapted to penetrate skin, tissue and/or a port septum of a port to transfer fluid into a port chamber of the port, the port needle comprising:
a) a distal section adapted to receive the fluid;
b) a proximal section adapted to deliver the fluid to the chamber of the port;
c) at least one lumen defining at least one inner surface, the lumen being adapted to transfer the fluid from the distal section to the proximal section; and
d) at least one membrane defining at least one outer surface of the proximal section;
e) wherein the outer surface of the proximal section comprises Ni and/or any Ni compound with a content of at most 0.05% and/or being adapted to release at most 0.2 μg/cm²/week of Ni and/or of any Ni compound.

2. The port needle according to claim 1, wherein the membrane is impermeable to liquids and/or gases.

3. The port needle according to claim 1, wherein the membrane is arranged as at least one outer layer on a substrate and/or is forming an outer wall at least of the proximal section.

4. The port needle according to claim 1, wherein the membrane is made of metal and/or metal alloy.

5. The port needle according claim 1, wherein the membrane comprises and/or is made of a non-metallic material.

6. The port needle according to claim 1, wherein the section modulus of the port needle is at least 0.004 mm³, preferably 0.01 mm³, more preferably 0.1 mm³, even more preferably 0.2 mm³ and/or at most 0.3 mm³, preferably 0.2 mm³, more preferably 0.1 mm³, even more 0.01 mm³.

7. The port needle according to claim 1, wherein the membrane is defined by a coating and/or a layer provided over at least the proximal section of the port needle, preferably adhered by a shrink fit and/or an adhesive.

8. The port needle according to claim 1, wherein the inner surface of the lumen of the port needle comprises Ni or any Ni compound of at most 0.05% and/or being adapted to release at most 0.2 μg/cm²/week of Ni or of any Ni compound.

9. The port needle according to claim 1, wherein the proximal section has a maximum length of 50 mm, preferably of 40 mm, more preferably of 30 mm, even more preferably of 20 mm, and even more preferably of 10 mm and/or a minimum length of 2 mm, preferably of 5 mm, more preferably of 10 mm, even more preferably of 20 mm, even more preferably of 30 mm.

10. The port needle according to claim 1, wherein the solid membrane and/or the membrane arranged as a layer together with the underlying substrate has/have a maximum outer diameter of 1.5 mm, preferably of 1.2 mm, more preferably of 1.0 mm, even more preferably of 0.9 mm, even more preferably of 0.8 mm, even more preferably of 0.7 mm and/or a minimum diameter of 0.3 mm, preferably of 0.4 mm, more preferably of 0.5 mm, even more preferably of 0.6 mm, even more preferably of 0.7 mm, even more preferably of 0.9 mm.

11. The port needle according to claim 1, wherein the proximal section of the port needle terminates in a blunt end, a bevelled end and/or an end with at least one lateral window.

12. The port needle according to claim 1, wherein the proximal section comprises a dull end and the port needle further comprises a piercing element with a sharp tip being invertible at least into the lumen so that the sharp tip extends out of the proximal section for penetrating the skin, tissue and/or port lumen and the piercing element being adapted to be taken out of the lumen.

13. A needle for use as a port needle according to claim 1.

14. Method A method of penetrating skin, tissue and/or a port septum of a port by a port needle to transfer fluid into a port chamber of the port, the method comprising the following steps:
a) receiving the fluid by a distal section;
b) delivering the fluid to the chamber of the port by a proximal section;
c) transferring the fluid from the distal section to the proximal section by at least one lumen defining at least one inner surface;
d) defining at least one outer surface of the proximal section by at least one membrane; and
e) providing the outer surface of the proximal section with a Ni and/or any Ni compound with a content of at most 0.05% and/or the outer surface being adapted to release at most 0.2 μg/cm²/week of Ni and/or of any Ni compound.

15. A method of penetrating a port septum of a port by a port needle to transfer fluid into a port chamber of the port, the method comprising:
a) providing the port outside of the human or animal body;
b) receiving the fluid by a distal section;
c) delivering the fluid to the chamber of the port by a proximal section;
d) transferring the fluid from the distal section to the proximal section by at least one lumen defining at least one inner surface;
e) defining at least one outer surface of the proximal section by at least one membrane; and
f) providing the outer surface of the proximal section with a Ni and/or any Ni compound with a content of at most 0.05% and/or the outer surface being adapted to release at most 0.2 μg/cm²/week of Ni and/or of any Ni compound.
